(19) United States
(12) Patent Application Publication
(10) Pub. No.: US 2012/0277882 A1
(43) Pub. Date: Nov. 1, 2012

(54) IMPLANT COMPOSITE PARTICLE, METHOD FOR MAKING THE SAME, AND USES THEREOF

(75) Inventors: Jo-Wei HUANG, Taoyuan Hsien (TW); Po-Yang Chen, Taipei City (TW); Ken-Yuan Chang, Taoyuan Hsien (TW)

(73) Assignee: FAR EASTERN NEW CENTURY CORPORATION

(21) Appl. No.: 13/457,610

(22) Filed: Apr. 27, 2012

(30) Foreign Application Priority Data
Apr. 29, 2011 (TW) 100115102

(51) Int. Cl. A61F 2/28; C08L 89/00; C08L 5/00; C08L 67/04

(52) U.S. Cl. 623/23.61; 523/116; 106/157.6; 106/217.9

(57) ABSTRACT

A biocompatible implant composite particle and the method of making the same are provided. The implant composite particle includes a bone filler particle and a plurality of fibers, in which each fiber is partially embedded in the bone filler particle, and has a free portion extending from a surface of the bone filler particle. Both bone filler particle and fibers are biocompatible. The biocompatible implant composite can be used in a bone filler material for bone defects.

### CROSS-REFERENCE TO RELATED APPLICATION

This application claims priority of Taiwanese application No. 100115102, filed on Apr. 29, 2011.

### BACKGROUND OF THE INVENTION

1. Field of the Invention

The present invention is directed to an implant composite particle and a preparation process thereof, more particularly to an implant composite particle comprising a bone filler particle and a plurality of fibers. This invention also relates to a bone filler material including the implant composite particle. 2. Description of the Related Art

Implantable bone filler materials are used to promote and aid the healing of bone defects. In general, there are two main categories of bone defects: one occurs at sites that do not need to bear too much load, such as the wrist and skull, whereas and the other occurs at sites that require support, such as the foot or spine. Bone filler materials used in the first category mainly emphasize on the resistance to degradation/decomposition caused by body fluid and requires less mechanical strength. The common way to repair the bone defect of the first category is to directly fill calcium phosphate powder into the sites of bone defect, or to use bone graft to rebuild a broken bone. Bone filler materials for the second category of bone defect require good mechanical strength and good resistance to body fluid, thereby providing a supporting function to a broken bone and preventing further damage.

U.S. Pat. No. 5,053,212 discloses a composition that is provided for the production of hydroxyapatite. Additives, such as bone associated proteins, e.g., collagen, may be added to provide a specific property, thereby obtaining a material that resembles physical properties of the bone. However, once the material is decomposed, the exposed protein additives might be scoured out and degraded by body fluid, hence losing its function.

U.S. Pat. No. 7,393,405 discloses a hydraulic cement for surgical use that is mainly composed of α-tricalcium phosphate powder particles, calcium sulphate dehydrate and water. Although calcium sulphate reinforces mechanical strength, it is likely to be absorbed by a human body after 6 months and will not be able to support the deficient bone.

From U.S. Pat. No. 6,783,712, it is known that a fiber—reinforced, polymeric implant material is useful for tissue engineering. The implant material comprises a polymeric matrix and fibers substantially uniformly distributed therein. The fibers are aligned predominantly parallel to each other. Although these fibers can increase mechanical strength of the polymeric matrix, the fibers distributed within the polymeric matrix might inevitably affect the compactness and mechanical strength of the polymeric matrix.

During the repair and healing process of the bone, the mere support provided by the bone filler material is inadequate. Additional features such as adhesion and proliferation of osteoblasts and secretion of extracellular matrix are required for the bone to reach full recovery. The most common bone filler material is polymethyl methacrylate. However, this polymer is not biodegradable, and cell attachment is less effective. Consequently, loose binding of the bone filler material and tissue cells leads to a brittle and fragile bone. Therefore, the main emphasis of the field is to find a filler material that can provide strong physical support and ideal physiological environment for osteoblasts growth.

### SUMMARY OF THE INVENTION

Therefore, according to the first aspect of this invention, an implant composite particle comprises a bone filler particle that is made from a biocompatible material, and a plurality of fibers each of which is composed of a biocompatible polymer, is partly embedded in the bone filler particle, and has a free portion extending from a surface of the bone filler particle.

In the second aspect of this invention, a method for making an implant composite particle comprises providing first and second solutions that are capable of producing a bone filler particle by acid-base reaction or cationic-anionic interaction, adding a fiber component including a plurality of fibers into at least one of the first and second solutions, and reacting the first and second solutions to form the bone filler particle with the fibers partially embedded therein.

In the third aspect of this invention, a bone filler material comprises the aforesaid implant composite particle.

### BRIEF DESCRIPTION OF THE DRAWING

Other features and advantages of the present invention will become apparent in the following detailed description of the preferred embodiments with reference to the accompany drawings, of which:

FIG. 1 is a schematic diagram showing the structure of the implant composite particle that comprises a bone filler particle and a plurality of fibers according to this invention.

### DETAILED DESCRIPTION OF THE PREFERRED EMBODIMENTS

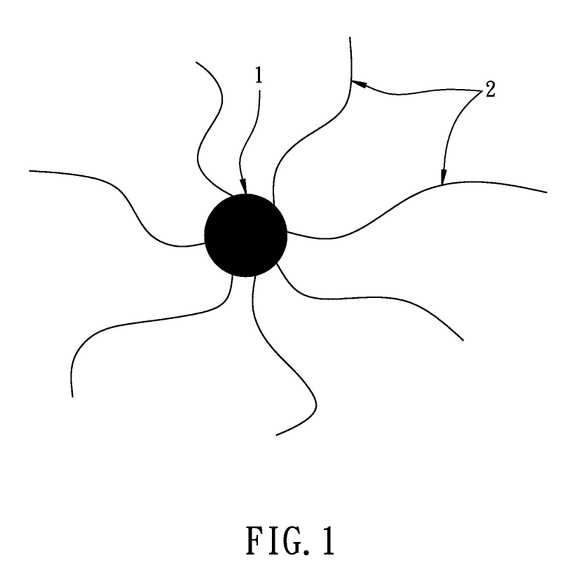

FIG. 1 shows an implant composite particle of the present invention which comprises a bone filler particle 1 and a plurality of fibers 2. The implant composite particle of this invention can be used to form a bone filler material, and thus, the present invention also provides a bone filler material that includes a plurality of the implant composite particles, in which the fibers of the implant composite particle are entangled with fibers of the adjacent bone filler particles.

The bone filler particle 1 has a diameter of 5 μm˜150 μm, and is made from a biocompatible material. Each of the fibers 2 is composed of a biocompatible polymer, and is partly embedded in the bone filler particle 1. Each of the fibers 2 has a free portion that extends from a surface of the bone filler particle 1 and the fibers have a length being one to twenty times of the diameter of the bone filler particle 1.

When the diameter of the bone filler particle 1 is smaller than 5 μm, the implant composite particle is likely to be phagocytosed by immune cells, thereby leading to the degradation of the implant composite particle. When the diameter of the bone filler particle 1 is larger than 150 μm, the relatively large particle size will result in large inter-particle spaces among the implant composite particles, and the bone filler material will have a loose structure. Preferably, the diameter of the bone filler particle 1 ranges from 10 μm to 100 μm, more preferably, from 20 μm to 50 μm.

When the length of the fiber 2 is more than twenty times of the diameter of the bone filler particle 1, the compactness of the bone filler material will be adversely affected, thereby resulting in a loose structure and weak mechanical strength. When the fiber length is less than the diameter of the bone filler particle 1, a less effective entanglement among the fibers 2 occurs, and the mechanical strength of the bone filler material is less augmented.

Preferably, the mean fiber length is 1.5 to 17.5 times longer than the diameter of the bone filler particle 1, and is more preferably 1.5 to 12 times longer than the diameter of the bone filler particle 1.

Preferably, the biocompatible polymer is selected from the group consisting of polysaccharide, polypeptide, polylactic acid, polyglycolic acid, polyethylene oxide, polyethylene glycol, polycaprolactone, polyvinyl alcohol, polyacrylic acid and combinations thereof.

Preferably, the polysaccharide is selected from the group consisting of chitosan, cellulose, alginate and combinations thereof.

Preferably, the polypeptide is selected from the group consisting of collagen, gelatin and a combination thereof. The preparation of the implant composite particle of the present invention is conducted: by providing first and second solutions that are capable of producing a bone filler particle by acid-base reaction or by cationic-anionic interaction; adding a fiber component including a plurality of fibers into at least one of the first and second solutions; and reacting the first and second solutions to form the bone filler particle with the fibers partially embedded therein. During reacting the first and second solutions, the fibers will be partially embedded in the bone filler particle.

In this invention, an example of the bone filler particle formed by acid-base reaction is calcium phosphate. In this case, the first solution includes calcium salt selected from the group consisting of calcium chloride, calcium carbonate, calcium nitrate, calcium hydroxide, calcium acetate, calcium gluconate, calcium citrate and combinations thereof. The second solution includes phosphate salt selected from the group consisting of tertiary potassium phosphate, monobasic sodium phosphate, disodium phosphate, trisodium phosphate, diammomium hydrogen phosphate, ammonium dihydrogen phosphate, triammonium phosphate, tetrasodium pyrophosphate, monopotassium phosphate, dipotassium hydrogen phosphate and combinations thereof.

In the case that the bone filler particle is produced by cationic-anionic interaction, the first solution includes a cationic material selected from the group consisting of chitosan, derivatives of chitosan and a combination thereof. The second solution includes an anionic material, e.g., anionic polypeptide and anionic polysaccharide. Examples of the anionic polypeptide include polyglutamic acid, derivatives of polyglutamic acid, polyaspartic acid and derivatives of polyaspartic acid. Examples of the anionic polysaccharide include alginate, cellulose and pectin.

The derivative of chitosan includes N-octyl-O, N-carboxymethyl chitosan.

The derivatives of polyglutamic acid and polyaspartic acid include salts thereof, such as magnesium salt, calcium salt, sodium salt, etc.

Bone filler materials must withstand physiological loads to support injured sites that require load bearing, such as shank bone and spine. Therefore, in addition to the implant composite particle, the bone filler material of this invention further includes calcium sulphate. The addition of calcium sulphate augments the mechanical strength of the bone filler material. In addition, entanglement of fibers of the implant composite particle secures calcium sulphate from being degraded/decomposed by body fluid, thereby maintaining a reinforced mechanical strength.

Preferably, the implant composite particle is present in an amount ranging from 5 wt % to 85 wt % based on the total weight of the bone filler material, more preferably, ranging from 10 wt % to 65 wt %. When the implant composite particle is less than 5 wt % of the bone filler material, entanglement of the fibers will be reduced, thereby leading to limited increase in mechanical strength. Since the mechanical strength is also provided by calcium sulphate, when the implant composite particle is more than 85 wt % of the bone filler material, the mechanical strength will be adversely affected.

The bone filler material of this invention may be used for bone defect caused by surgery, injury, etc.

### EXAMPLES

This invention will be further described by way of the following examples. However, it should be understood that the following examples are solely intended for the purpose of illustration and should not be construed as limiting the invention in practice.

### <Source of Chemicals>

1. Collagen: purchased from Sigma; catalog number: Bornstein and Traub Type I (Sigma Type III).
2. 1,1,1,3,3,3 hexafluoro-2-propanol: purchased from Fluka, purity: ≧99.0%.
3. Chitosan: purchased from Aldrich.
4. Trifluoroacetic acid: purchased from Sigma; Catalog number: ReagentPlus®; purity: 99%
5. Polyglutamic acid: purchased from Vedan, catalog number: Na form
6. Polycaprolactone: purchased from Aldrich; weight average molecular weight (M_{w}): about 65,000°
7. Hydroxyapatite: purchased from sigma; purity: ≧99.0%

### Experimental Materials:

### [Preparation of Collagen Fiber]:

The collagen fiber used herein was made by the inventors of this invention. 0.3 g of collagen was dissolved in 5 mL of 1,1,1,3,3,3 hexafluoro-2-propanol to obtain a 6 wt % collagen solution. The solution was subjected to an electrospinning process so as to obtain a mesh of fine fibers. In the electrospinning process, a voltage was 20 kV, and the distance between a needle tip where a jet was erupted and a grounded collector was 7 cm. The mesh was subjected to refrigeration milling process. The collagen fiber length was determined by controlling the frequency of the refrigeration milling process.

### [Preparation of Chitosan Fiber]:

The chitosan fiber used in the examples below was made by the inventors of this invention. 0.35 g of chitosan was dissolved in 5 mL of 1,1,1,3,3,3 hexafluoro-2-propanol to obtain a 7 wt % chitosan solution. The solution was subjected to an electrospinning process to obtain a mesh of fine fibers. In the electrospinning process, a voltage was 20 kV, and the distance between a needle tip where a jet was erupted and a grounded collector was 5 cm. The mesh was subjected to refrigeration milling process. The chitosan fiber length was determined by controlling the frequency of the frozen grinding process.

### [Preparation of Polycaprolactone Fiber]:

The polycaprolactone fiber used in the examples below was made by the inventors of this invention. 0.25 g of polycaprolactone was dissolved in 5 mL of 1,1,1,3,3,3 hexafluoro-2-propanol to obtain a 5 wt % polycaprolactone solution. The solution was subjected to an electrospinning process to obtain a mesh of fine fibers. In the electrospinning process, a voltage was 18 kV, and the distance between a needle tip where a jet was erupted and a grounded collector was 4 cm. The mesh was subjected to refrigeration milling process. The polycaprolactone fiber length was determined by controlling the frequency of the refrigeration milling process.

### Preparation of Implant Composite Particle

### Example 1

0.5 g of the aforementioned collagen fiber (average fiber length: 240 μm) was evenly dissolved in 14 mL of 0.1 M calcium chloride to form a mixture. 4.2 mL of 0.1 M disodium phosphate was slowly added into the mixture, followed by adjusting pH to 7.0 using 0.1 M NaOH solution. After 1 hr of stirring, a precipitate was obtained by three times of centrifugation and washed with deionized water followed by lyophilization. Implant composite particles having an average diameter of 20 μm were obtained.

### Example 2

0.7 g of the aforementioned chitosan fiber (average fiber length: 400 μm) was evenly dissolved in 14 mL of 0.1 M calcium chloride solution to form a mixture. 8.4 mL of 0.1 M disodium phosphate solution was slowly added into the mixture, followed by adjusting pH to 7.0 using 0.1 M NaOH solution. After 1 hr of stirring, a precipitate was obtained by three times of centrifugation and washed with deionized water followed by lyophilization. Implant composite particles having an average diameter of 50 μm were obtained.

### Example 3

2 g of the aforementioned chitosan fiber (average fiber length: 40 μm) was evenly dissolved in 20 mL of 10 wt % polyglutamic acid solution to form a mixture. 20 mL of 2 wt % chitosan solution was slowly added into the mixture, followed by adjusting pH to 7.0 using 0.1 M NaOH solution. After 1 hr of stirring, a precipitate was obtained by three times of centrifugation and washed with deionized water followed by lyophilization. Implant composite particles having an average diameter of 20 μm were obtained.

### Example 4

2.0 g of the aforementioned polycaprolacton fiber (average fiber length: 40 μm) was evenly dissolved in 20 mL of 10 wt % polyglutamic acid solution to form a mixture. 20 mL of 2 wt % chitosan solution was slowly added into the mixture, followed by adjusting pH to 7.0 using 0.1 M NaOH solution. After 1 hr of stirring, a precipitate was obtained by three times of centrifugation and washed with deionized water followed by lyophilization. Implant composite particles having an average diameter of 20 μm were obtained.

### Examples 5 and 6

The process in each of Examples 5 and 6 was similar to that of Example 1, except that, in Examples 5 and 6, the average fiber lengths of the collagen fibers were 30 μm and 350 μm, respectively.

### Comparative Example 1

4.2 mL of 0.1 M disodium phosphate solution was slowly added into 14 mL of 0.1 M calcium chloride solution, followed by adjusting pH to 7.0 using 0.1 M NaOH solution. After 1 hr of stirring, the precipitate was obtained by three times of centrifugation and washed with deionized water, followed by lyophilization. Calcium phosphate particles having an average diameter of 20 μm were obtained.

### Comparative Example 2

20 mL of 2 wt % chitosan solution was slowly added to 20 mL of 10% polyglutamic acid solution followed by adjusting pH to 7.0 using 0.1 M NaOH solution. After 1 hr of stirring, a precipitate was obtained by three times of centrifugation and washed with deionized water, followed by lyophilization. The polyglutamic acid-chitosan particles having an average diameter of 20 μm were obtained.

### Entanglement Test:

Entanglement tests for the implant composite particles were used to determine the resistance of the implant composite particles to washing-away by fluid. 1 g of implant composite particles of examples 1-6 and the particles of comparative examples 1-2 were pressed into round plates with 8 mm diameter and 2 mm thickness. These round plates were flushed with water expelled from a syringe. Results are shown in Table 1. O: that the sample remains in round plate form. X: indicates that the sample is decomposed.

TABLE 1

| | | | Fiber | | |
|---|---|---|---|---|---|
| | Implant composite | | | Mean | |
| | particle | | | fiber | Entan- |
| | | Diameter | | length | glement |
| | composition | (μm) | material | (μm) | test |
| Example 1 | Calcium | 20 | collagen | 240 | ◯ |
| | Chloride + | | | | |
| | Disodium | | | | |
| | phosphate | | | | |
| Example 2 | Calcium | 50 | chitosan | 400 | ◯ |
| | Chloride + | | | | |
| | sodium | | | | |
| | dihydrogen | | | | |
| | phosphate | | | | |
| Example 3 | polyglutamic | 20 | chitosan | 40 | ◯ |
| | acid + | | | | |
| | chitosan | | | | |
| Example 4 | polyglutamic | 20 | poly- | 40 | ◯ |
| | acid + | | caprolactone | | |
| | chitosan | | | | |
| Example 5 | Calcium | 20 | collagen | 30 | ◯ |
| | Chloride + | | | | |
| | Disodium | | | | |
| | phosphate | | | | |
| Example 6 | Calcium | 20 | collagen | 350 | ◯ |
| | Chloride + | | | | |
| | Disodium | | | | |
| | phosphate | | | | |
| Comparative | Calcium | 20 | None | None | X |
| Example 1 | Chloride + | | | | |
| | Disodium | | | | |
| | phosphate | | | | |
| Comparative | polyglutamic | 20 | None | None | X |
| Example 2 | acid + | | | | |
| | chitosan | | | | |

As shown in Table 1, the particles of comparative examples 1 and 2 were washed away by water, which suggests that the particles of comparative examples 1 and 2 have low structural compactness. However, each of the samples of Examples 1 to 6 remains in a round plate form. This is due to the entangled fibers formed among the implant composite particles of this invention, thereby providing a structural compactness that is sufficient to maintain its integrity after applying water force.

### Preparation Sample of Bone Filler Material

### Example 7

The bone filler material of this current example was derived from a combination of the implant composite particle of example 1 with calcium sulphate at a weight ratio of 1:9 and in the form of powder. 5 g of the bone filler material powder was added to 2.5 mL of saline (purchased from Sin Tong, Taiwan) and stirred for at least one minute to obtain a sample of the bone filler material.

### Examples 8 to 10

The preparation method for the sample of the bone filler material in each of Examples 8 to 10 was the same as that in the aforementioned Example 7. The only difference was the implant composite particles used in Examples 8 to10 were from Examples 3, 5 and 6 respectively.

### Examples 11 to 13

The preparation method for the sample of the bone filler material in each of Examples 11 to13 was the same as that in the aforementioned Example 7. The only difference was the weight ratios of the implant composite particles to calcium sulphate were 1:12, 1.9:1 and 9:1 respectively.

### Comparative Example 3

The bone filler material in Comparative Example 3 was obtained by mixing hydroxylapatite with calcium sulphate in 1:1 ratio (by weight). 5 g of the bone filler material powder was added to 2.5 mL of saline (purchased from Sin Tong, Taiwan) and stirred for at least one minute to obtain a sample of the bone filler material.

### Comparative Example 4

Collagen was dissolved in 0.1M acetic acid in order to obtain a 3 wt % collagen solution. 2.5 g of hydroxylapatite and 2.5 g of calcium sulphate (weight ratio of 1:1) was added into 2.5 mL of collagen solution and evenly mixed for at least 1 hr. A sample of the bone filler material was obtained.

### Comparative Example 5

A sample of the bone filler material was obtained by mixing hydroxylapatite, calcium sulphate and the aforementioned collagen fiber (average length 240 μm) at a weight ratio of 1:1:0.2.

### Comparative Examples 6 to 7

The preparation method for the sample of the bone filler material in each of Comparative Examples 6 to 7 was the same as in the aforementioned Example 7. The only difference was the implant composite material used in Comparative examples 6 to 7 were from Comparative Examples 1 and 2, respectively. Strength test of the sample of the bone filler material

Each of the samples in the aforementioned Examples 7 to 13 and Comparative Examples 3 to 7 was placed into a cylindrical mold having a radius of 6 mm and a height of 12 mm before solidification. Each of the samples was allowed to be solidified under an ambient temperature of 37° C. for 24 hrs and was taken out of the mold to obtain a cylindrical sample. The cylindrical sample was then immersed in water and subjected to ultrasonic vibration for seven days. A material testing machine (purchased from: PRO TEST, model number: PT-1066) was used to determine compression stress of the cylindrical samples before and after immersing in water. The compression velocity was 1 mm/min. The results are shown in Table 2.

TABLE 2

| | | | | | Change |
|---|---|---|---|---|---|
| | | | | | rate in |
| | | Weight | | | com- |
| | | ratio | | | pression |
| | | of the | | | stress |
| | | implant | | | before |
| | | com- | compression | | and |
| | | posite | stress (MPa) | | after |
| | | particle | Before | After- | im- |
| | Implant | to | im- | im- | mersing |
| | composite | calcium | mersing | mersing | in |
| | particle | sulphate | in water | in water | water (%) |
| Example 7 | Example 1 | 1:9 | 45.6 | 40.2 | 11.84% |
| Example 8 | Example 3 | 1:9 | 41.5 | 35.0 | 15.66% |
| Example 9 | Example 5 | 1:9 | 41.9 | 26.8 | 36.04% |
| Example 10 | Example 6 | 1:9 | 30.3 | 22.4 | 26.07% |
| Example 11 | Example 1 | 1:12 | 49.7 | 31.1 | 37.42% |
| Example 12 | Example 1 | 1.9:1 | 5.2 | 4.5 | 13.46% |
| Example 13 | Example 1 | 9:1 | — | — | — |
| Comparative | Hydroxylapatite | 1:1 | 41.7 | 20.6 | 50.60% |
| Example 3 | | | | | |
| Comparative | Hydroxylapatite | 1:1 | 50.8 | 10.2 | 79.92% |
| Example 4 | | | | | |
| Comparative | Collagen fiber | 1.2:1 | 35.4 | 20.1 | 43.22% |
| Example 5 | (240 μm) + | | | | |
| | Hydroxylapatite | | | | |
| Comparative | Comparative | 1:9 | 44.0 | 19.6 | 55.45% |
| Example 6 | Example 1 | | | | |
| Comparative | Comparative | 1:9 | 40.9 | 23.1 | 43.52% |
| Example 7 | Example 2 | | | | |

“—” indicates no measurement

As shown in Table 2, compression stress decreased in all the samples after seven days of immersing in water. This suggests that the samples will be gradually decomposed under a humid environment, thereby leading to a change in its properties.

The change rate in compression stress before and after immersing in water exceeds 40% in samples obtained from Comparative Examples 3 to 7. The sample of the bone filler material form Comparative Example 3 composed of a combination of hydroxylapatite and calcium sulphate has a decreased compression stress of about 50%. Although there is good compression stress in Comparative Example 4 before immersing in water, the compression stress decreased about 80% after immersing in water. When using collagen (a thickening agent) as a base, the interspaces among hydroxylapatite particles could be filled with collagen, therefore providing the best compression stress before immersing in water. However, the collagen is gradually leached out after immersing in water, therefore leading to decreased compression stress. Sample from Comparative Example 5 has a lower compression stress before immersing in water when compared to the sample obtained from Comparative Example 3. This may be due to loose structural density caused by the collagen fibers. However, because of entanglement of the fibers after immersing in water, a lower compression stress change is achieved in Comparative Example 5 when compared to Comparative Example 3.

The compression stress changes in samples from Examples 7 to 12 were all less than 40%. Compression stress change in samples from Examples 7 to 8 was less than 20% after 7 days of immersion and ultrasonic vibration in water. Although immersion and vibration in water will cause structural damage, the entangled fibers among the implant composite particles lead to a structural reinforcement and less decomposition. The larger compression stress change in Example 7 when compared to Examples 9 and 10 suggests that lengthy fibers lead to destruction of the structural density, thus resulting in decreased mechanical strength. In contrast, when the fiber length is too short, the fibers can not entangle effectively, and are less helpful for the reinforcement of mechanical strength.

The samples obtained from Example 11 had a higher compression stress before immersing in water due to the higher content of calcium sulphate. However, the low content of the implant composite particles results in less contact and entanglement of the fibers, thereby leading to lower mechanical strength after immersing in water. However, Examples 7 to 11 have better compression stress after immersing in water for seven days when compared to the comparative examples.

Compression stress was not tested in example 13. However, since it maintains a certain structure after immersing in water, this suggests that the fibers among the implant composite particles are entangled. Therefore, this material could be applied at sites that do not require load-bearing functions.

### Biological Test

The inventors used {3-[4,5-Dimethylthiazol-2-yl]-2,5-diphenyltetrazolium bromide} (MTT assay) to measure viability and proliferation of cells. Implant composite particles from each of Examples 1 to 4 were placed in a well of a 96-well plate, and were slightly compressed. The sample from each of Examples 7 and 8 and Comparative Examples 3 and 4 was also placed in a well of the 96-well plate. 1×10⁴ of L-929 mouse fibroblast cells (purchased from Bioresource Collection and Research Center (BCRC) of Food industry Research and Development Institute (FIRDI), catalog number: BCRC 60091) in 200 μL of medium were added into each well and incubated for 24 hrs at 37° C. Subsequently, supernatant was removed and 20 μL of MTT solution (dissolved in phosphate buffered saline (PBS) to a concentration of 5 mg/mL) was added into each well and the 96-well plate was covered with foil to avoid exposure to light. After 5 hrs of incubation, supernatant was removed and 200 μL of dimethyl sulfoxide (DMSO) was added in each well, followed by mixing uniformly at 100 rpm for 5 minutes to obtain a mixture. ELISA reader scanning multi-well spectrophotometer (purchased from BIOTEK, catalog number: POWERWAVE XS) was used to measure the absorbance of the mixture at 630 nm. The absorbance correlates to the number of viable cells. Absorbance lower than 0.5 is an indication of non-ideal cell growth. The results are shown in Table 3.

TABLE 3

| | Absorbance |
|---|---|
| Example 1 | 0.907 |
| Example 2 | 0.882 |
| Example 3 | 0.710 |
| Example 4 | 0.647 |
| Example 7 | 0.866 |
| Example 8 | 0.575 |
| Comparative Example 3 | 0.286 |
| Comparative Example 4 | 0.491 |

As shown in Table 3, the implant composite particle from each of Examples 1 to 4 provides an environment beneficial for cell growth, with particles having collagen fibers as in Example 1 the most ideal. Cell growth on the sample of the bone filler material obtained from Examples 7 or 8 are better than that obtained from Comparative Example 3. This indicates that the implant composite particle provided with the fibers can promote cell adhesion and growth. The absorbance in Comparative Example 4 does not reach the same value as in Examples 7 and 8. This may be due to the dissolution of the collagen from the sample into the medium.

To sum up, in this present invention, the implant composite particle used in the bone filler material has a special structure, i.e., a bone filler particle with a plurality of fibers and the fibers among the particles are entangled together, thus making the bone filler material resistant to degradation or washing-away by body fluid. The biocompatible polymer used to make the fiber and bone filler particle of the implant composite particle promotes cell adhesion and growth and has good compatibility with cells.

While the present invention has been described in connection with what are considered the most practical and preferred embodiments, it is understood that this invention is not limited to the disclosed embodiments but is intended to cover various arrangements included within the spirit and scope of the broadest interpretation and equivalent arrangements.

What is claimed is:

1. An implant composite particle, comprising:
a bone filler particle made from a biocompatible material having a particle diameter ranging from 5 μm-150 μm, and
a plurality of fibers each of which is composed of a biocompatible polymer, is partly embedded in said bone filler particle, and having a length which is one to twenty times of said particle diameter of said bone filler particle.

2. The implant composite particle according to claim 1, wherein said biocompatible polymer is selected from the group consisting of polysaccharide, polypeptide, polylactic acid, polyglycolic acid, polyethylene oxide, polyethylene glycol, polycaprolactone, polyvinyl alcohol, polyacrylic acid and combinations thereof.

3. The implant composite particle according to claim 2, wherein said polysaccharide is selected from the group consisting of chitosan, cellulose, alginate and combinations thereof.

4. The implant composite particle according to claim 2, wherein said polypeptide is selected from the group consisting of collagen, gelatin and a combination thereof.

5. The implant composite particle according to claim 1, wherein said bone filler particle is calcium phosphate.

6. The implant composite particle according to claim 1, wherein said bone filler particle is composed of an anionic material and a cationic material.

7. The composite particle according to claim 6, wherein said anionic material is selected from the group consisting of polyglutamic acid, derivatives of polyglutamic acid, polyaspartic acid, derivatives of polyaspartic acid, alginate, cellulose, pectin and combinations thereof.

8. The composite particle according to claim 6, wherein said cationic material is chitosan or derivatives thereof.

9. A method for making an implant composite particle comprising:
a. providing first and second solutions that are capable of producing a bone filler particle by acid-base reaction or by cationic-anionic interaction;
b. adding a fiber component including a plurality of fibers into at least one of the first and second solutions; and
c. reacting the first and second solutions to form the bone filler particle with the fibers partially embedded therein.

10. The method according to claim 9, wherein the first solution includes a calcium salt selected from the group consisting of calcium chloride, calcium carbonate, calcium nitrate, calcium hydroxide, calcium acetate, calcium gluconate, calcium citrate and combinations thereof, and the second solution includes a phosphate salt selected from the group consisting of tertiary potassium phosphate, monobasic sodium phosphate, disodium phosphate, trisodium phosphate, diammomium hydrogen phosphate, ammonium dihydrogen phosphate, triammonium phosphate, tetrasodium pyrophosphate, monopotassium phosphate, dipotassium hydrogen phosphate and combinations thereof.

11. The method according to claim 9, wherein the first solution includes a cationic material, and the second solution includes an anionic material.

12. The method according to claim 11, wherein the cationic material is selected from the group consisting of chitosan, derivatives of chitosan and the combination thereof, and the anionic material being selected from the group consisting of polyglutamic acid, derivatives of polyglutamic acid, polyaspartic acid, derivatives of polyaspartic acid, alginate, cellulose, pectin and combinations thereof.

13. The method according to claim 9, wherein the fiber component is made from a biocompatible polymer.

14. The method according to claim 13, wherein the biocompatible polymer is selected from the group consisting of polysaccharide, polypeptide, polylactic acid, polyglycolic acid, polyethylene oxide, polyethylene glycol, polycaprolactone, polyvinyl alcohol, polyacrylic acid and combinations thereof.

15. The method according to claim 14, wherein the polysaccharide is selected from the group consisting of chitosan, cellulose, alginate and combinations thereof.

16. The method according to claim 14, wherein the polypeptide is selected from the group consisting of collagen, gelatin and a combination thereof.

17. A bone filler material comprising the implant composite particle of claim 1.

18. The bone filler material according to claim 17, further comprising calcium sulphate.

19. The bone filler material according to claim 17, wherein, based on the total weight of said bone filler material, the implant composite particle is present in an amount ranging from 5 wt % to 85 wt %.
